(19) United States
(12) Patent Application Publication
(10) Pub. No.: US 2006/0127873 A1
(43) Pub. Date: Jun. 15, 2006

(54) Composition for cytocompatible, injectable, self-gelling chitosan solutions for encapsulating and delivering live cells or biologically active factors

(76) Inventors: Caroline Hoemann, Montreal (CA); Abdellatif Chenite, Kirkland (CA); Michael Buschmann, Montreal (CA); Alessio Sarreqi, Montreal (CA); Jun Sun, Montreal (CA)

Correspondence Address: David S Resnick;Nixon Peabody, 100 Summer Street, Boston, MA, 02110-2131, US

(21) Appl. No.: 10/521,524

(22) PCT Filed: Jul. 16, 2003
(86) PCT No.: PCT/CA03/01069

§ 371 Date: Sep. 20, 2005

Related U.S. Application Data
(60) Provisional application No. 60/395,991, filed on Jul. 16, 2002.

(51) Int. Cl. A01N 1/02; C12N 5/06; A61K 31/717

(52) U.S. Cl. 435/2; 435/325; 514/55; 514/57

(57) ABSTRACT

The present invention provides compositions and methods for tissue repair using a cytocompatible self-gelling cross-linked hydrogel. The composition comprises a biocompatible mixture of chitosan, bifunctional dialdehyde, and hydroxylated polymer, which can be used to immobilize or encapsulate viable cells, or bioactive substances. The method includes the process of mixing bioactive substances, live cells, and/or extracellular matrix components with a cross-linking solution comprising a bifunctional aldehyde-treated hydroxylated polymer such as hydroxyethyl cellulose. The cross-linking solution is then mixed homogenously with a neutral isotonic chitosan solution. The chitosan becomes cross-linked by the bifunctional aldehyde, while the cells are protected from potentially nocive effects of the aldehyde cross-linker by the hydroxylated polymer. The injectable solution retains cell viability and bioactivity, and immobilizes cells at the site of injection or delivery. Depending on the particular application, mixtures of chitosan and bifunctional dialdehyde may be employed. The injectable solution also liberates bioactive substances with controlled release kinetics from the site of injection.

### BACKGROUND OF THE INVENTION

The invention relates to a composition and method of application to encapsulate live cells with a neutral isotonic chitosan gel solution that is able to solidify in situ with the aid of a cytocompatible cross-linker to aid tissue regeneration or wound-healing.

### DESCRIPTION OF PRIOR ART

1) Chitosan Liquid Solutions

Chitosan with a degree of deacetylation (DDA) between 50% DDA and 100% DDA can be completely solubilized in acidic aqueous solutions having a pH below the apparent chitosan pKa (pH 2.5 to pH 6.0). Such chitosan solutions are incompatible with cell viability. Attempts to raise the pH to cytocompatible levels with most buffers will cause the solution to precipitate, unless as shown previously by the Applicant, the buffer used is a polyol-phosphate (glycerol phosphate, GP) dibasic salt (Chenite Patent publication WO 99/07416). Chitosan/GP liquid solutions of pH 6.8 to 7.2 are cytocompatible and thermo-gelling. However, Chitosan/GP solutions capable of gelling at temperatures near body-temperature contain salt concentrations well beyond cytocompatible limits (8% disodium-GP is ˜360 mM, or 1080 mOsm). The thermogelling temperature is inversely proportional to the GP concentration, such that lowering the GP concentration to isotonic levels of salt (3% disodium-GP, ˜126 mM, 378 mOsm) results in a solution that is thermogelling at non-physiological temperatures, above 65° C. Therefore, cytocompatible liquid chitosan solutions may be generated using acid-solubilized chitosan brought to cytocompatible pH and tonicity with GP, however these solutions are unable to gel in an open body cavity or petri.

2) Cross-Linked Gels from Liquid Chitosan Solutions.

Many chemical cross-linkers have been proposed to form solid gels from liquid chitosan, including glyoxal (Freeman U.S. Pat. No. 5,489,401, 1996), glutaraldehyde (Hsien T-Y and Rorrer G L Ind Eng Chem Res. 36: 3631-3638, 1997; Oyrton A C Montiero Jr and Airoldi C. Internat. J. Biological Macromolecules. 26:119-128, 1999; Kumbar, S G, et al., J. Microencapsulation 19: 173-180, 2002; and Mi, F-L, et al., Biomaterials. 23:181-191, 2002), squarate (DeAngelis A A, et al., Macromolecules 31:1595-1601, 1998), oligo(ethylene oxide) (Rogovina S Z, et al., Polymer Science, 43: 265-268, 2001), tetramethoxy propane (Capitani D, et al., Carbohydrate Polymers 45:245-252, 2001), and genepin (Mi, F-L, et al., Biomaterials. 23:181-191, 2002). A prior invention has also taught that neutral chitosan solutions may be induced to gel using glyoxal solutions between 0.01% and 10% by weight glyoxal or other bifunctional cross-linker (Chenite et al. WO02/40070). However, these concentrations of glyoxal are toxic to cells.

A method for entrapping live cells in a chitosan gel using pH-dependent precipitation and non-covalent cross-links, in contrast to a chemical cross-linker, has been previously invented (Aebischer et al., U.S. Pat. No. 5,871,985). However this pH-dependent gellation mechanism leads to a form of chitosan paste that lacks the adhesive and mechanical properties of the chitosan gels described herein, and would have limited use in the domain of cartilage repair applications where the gel is to be applied to a tissue surface in an open body cavity such as the synovial joint.

In a previous invention (WO02/00272) a cytocompatible chitosan-GP liquid solution was proposed for use in cell encapsulation for tissue repair or regeneration based on thermogelling properties of the liquid chitosan-GP solution. Retention of viable cells in a solid chitosan gel with a composition of chitosan-GP, glucosamine, and hydroxyethyl cellulose was described. In a separate publication (Li and Xu, J. pharm. Sci. 91(7): 1669-1677, 2002), hydroxyethyl cellulose was proposed as a cytocompatible cross-linker of neutral chitosan-GP gels for cell encapsulation through a proposed mechanism of hydrogen bonding. The present invention is completely distinguished from these previous descriptions, by teaching a method and composition to encapsulate live cells using glyoxal-based cross-linking mechanism of chitosan-GP solutions that results in retention of viable cells in solidified gels.

In summary of prior art, acid-chitosan solutions have been cross-linked with an array of bifunctional cross-linkers with no evidence that these gelling solutions are able to maintain cell viability. Therefore, there is presently a lack of evidence concerning encapsulation of viable cells in chemically cross-linked chitosan gels,

It would be highly desirable to be provided with a new composition for use in medical contexts of tissue repair and regeneration.

### SUMMARY OF THE INVENTION

One aim of the present invention is to provide a biocompatible polymeric liquid solution loaded with cells or biologically active factors, which can solidify and form an implant or film with entrapped or immobilized cells or factors. The solution can thus form a biocompatible solid scaffolding that sustains cell viability, or offers controlled release of bioactive molecules at the injection site. After injection, the implant may give a therapeutic effect from delivered cells, hormones, drugs, DNA, or bulking agent.

In accordance with the present invention, there is provided a composition for immobilizing and encapsulating viable and functional cells or bioactive substances comprising:
a) a liquid polysaccharide solution of isotonic neutral chitosan; and
b) a cross-linking solution consisting of a bifunctional or multifunctional, aldehyde or aldehyde-treated hydroxyl-containing polymer dissolved in physiological media.

The cross-linking solution preferably consists of a bifunctional or multifunctional cross-linker and a hydroxylated polymer of appropriate ratio and molecular mass such as to permit the hydroxylated polymer to remain liquid in solution.

The cross-linking solution more preferably consists of glyoxal, or glyoxal-treated hydroxyethyl cellulose dissolved in physiological media.

The chemical cross-linker is preferably dissolved in physiological media harboring one more more cell nutrients including but not limited to glucose, vitamins, amino acids, and buffering agents as are found in typical cell culture media.

The composition of the present invention may comprise for example:
a) 0.5 to 5.0% by weight chitosan, or chitosan derivative, or poly-amine containing polymer; and
b) 0.0001-3% glyoxal,
and optionally
c) 0.01 to 5.0% by weight hydroxyethyl cellulose; and
Wherein said solution form a gel between temperatures of 4° C. and 42° C., and more preferably between 20° C. and 42° C., said gel providing a physiological environment for maintaining viability of cells.

The composition forms a gel, preferably within seconds to several hours after mixing (a) and (b), and (c) if present.

The chitosan is preferably dissolved in dilute acid and mixed with 1.0 to 2.5% by weight of a salt of polyol consisting of mono-phosphate dibasic salt, such as mono-phosphate dibasic salt of glycerol like glycerol-2-phosphate dibasic salt, sn-glycerol 3-phosphate dibasic salt and L-glycerol-3-phosphate dibasic salt, or mono-sulfate salt.

The chitosan may further be mixed with phosphate buffer and salt.

In one embodiment of the invention, the composition further comprises a biologically active factor. Such factor may be for example selected from the group consisting of cells, a hormones, a drug, DNA, a bulking agent, a growth factors, a DNA, DNA-polymer complexes, liposomes, a pharmacological agent, a metabolic factor, an antibody, a nutritive factor, an angiogenic factor, and a radioisotope.

In one embodiment of the invention, the composition is loaded with cells and more preferably live cells. The cells can be nucleus pulpopus, annulus fibrosis, or a mixture thereof. Alternatively, the cells can be embryonic stem cells or stem cells derived from a tissue selected from the group consisting of bone marrow, adipose, muscle, brain, skin, liver, vascular smooth muscle, endothelium, blood, or placenta. In fact, the cells could also be primary cells, differentiated cells, genetically modified cells, hybridomas, immortalized cells, transformed cells, tissue fragment cells, organelles, or a mixture thereof, nucleated cells, enucleated cells, germ cells, platelet cells, matrix vesicles, cell vesicles, demineralized bone paste, bone chips, cartilage fragments, or cell fragments or tissue fragments, as well as autologous cells, allogeneic cells or xenogeneic cells.

In one embodiment of the invention, the biologically active factor is a cell attachment factor selected from the group consisting of fibrinogen, fibrin, fibronectin, hyaluronic acid, heparin, collagen, polylysine, polyornithine, receptor-binding cyclic peptide, and receptor-binding protein.

The biologically active factor can also be an enzyme, a growth-factor or a growth factor-immobilized substance, as well as a plasmid DNA in the form of liposomes, a lipid complex, a chitosan complex, a poly-lysine complex, a DEAE dextran complex.

In a preferred embodiment, the biologically active factor is a vaccine, either for active or passive immunization. The vaccine can thus comprise an infective viral particle.

The biologically active factor can also be a nutritive or metabolic factor such as a lipid, amino acids, and a co-factor selected from the group consisting of cholesterol, glutamine, glucosamine, ascorbic acid, pyruvate, and lactate.

The biologically active factor can further be at least one element selected from the group consisting of peripheral blood, bone blood, cord blood, a blood product, blood-borne cells, serum, platelets, platelet-rich plasma, fibrinogen, a clotting factor, and a blood-borne enzyme.

In one embodiment of the invention, the biologically active factor is an osteogenic substance such as a member of the bone morphogenetic protein family selected from the group consisting of TGF-01, BMP-2, BMP-6, BMP-7, or a mixture thereof.

In one embodiment of the invention, the hydroxyl-containing polymer is polyvinyl alcohol, dextran, linked with a bifunctional reactive aldehyde.

Still in accordance with the present invention, there is provided the use of the composition of the present invention for soft tissue repair, for site-specific delivery of said biologically active factor, for bone repair, for repairing or resurfacing damaged cartilage or for repairing meniscus. In accordance with the present invention, there is also provided the use of the composition of the present invention for the manufacture of a medicament for the various use mentioned herein.

Of course, one skilled in the art provided with the composition of the present invention, and being told that the composition can be used for the various uses mentioned herein will have no difficulty using the composition in a method of treatment. Accordingly, these methods are also included in the present invention.

In the present application, the expression biologically active factors” is meant to include without limitation any biologically active ingredients, cells that have a therapeutic effect, hormones, drugs, DNA, bulking agent, growth factors, DNA, DNA-polymer complexes, liposomes, pharmacological agents, metabolic factors, antibodies, nutritive factors, angiogenic factors, or radioisotopes etc . . . .

### BRIEF DESCRIPTION OF THE DRAWINGS

FIG. 1A illustrates the method used to generate cytocompatible cross-linker by cross-linking hydroxyethyl cellulose with glyoxal;

FIG. 1B illustrates an example of a mechanism of gellation by mixture of glyoxal-cross-linked hydroxyethyl cellulose with chitosan;

FIG. 1C illustrates various methods for preparing cytocompatible cross-linker;

FIGS. 2A to 2C illustrate spectral characterization of active and inactive cross-linker prepared by the method illustrated in FIG. 1C;

FIGS. 2D to 2G demonstrate gelation over six minutes after mixing;

FIG. 3 illustrates the evolution of G′ and G″ with time at room temperature (25° C.) for a typical cross-linked formulation comprising the successive mixture of 0.12 g chitosan (76% DDA) dissolved in 9 ml 67 mM HCl solution, 0.41 g b-glycerol phosphate dissolved in 1 ml ddH₂0, and 3 to 30 mg water-soluble Spectrum reagent-grade hydroxyethyl cellulose dissolved in 2 ml buffered Ringer's Lactate solution;

FIG. 4A illustrates viability of cells maintained in hydroxyethyl cellulose, or glyoxal cross-linker for over an hour;

FIG. 4B shows viability of cells (MTT assay for live cell metabolism) and cell proliferation (Hoechst DNA assay to reflect cell density) in chitosan gels cross-linked with glyoxal or hydroxyethyl cellulose-glyoxal;

FIGS. 5A and 5B illustrate viability of various cell types in chitosan gel cross-linked with hydroxyethyl cellulose/aldehyde (FIG. 5A), or glyoxal (FIG. 5B);

FIG. 5C illustrates comparable viability in 2% low melting agarose gel;

FIG. 6 shows examples of typical compositions of cross-linked cytocompatible chitosan gels using hydroxyethyl cellulose-glyoxal, or glyoxal, used to encapsulate viable cells, in accordance with the present invention;

FIGS. 7A to 7C show examples of cell delivery applications in cartilage repair using neutral cross-linked chitosan gels using hydroxyethyl cellulose-glyoxal or glyoxal, depending on the application;

FIGS. 8A and 8B shows the persistence cross-linked chitosan gel in vivo, in rabbit articular or osteochondral defects from 1 day, to 30 days post-injection; and

FIGS. 9A and 9B show the formation of neocartilage tissue in vitro (FIG. 9A) and in vivo (FIG. 9B) when primary chondrocytes are encapsulated in cross-linked chitosan gel using hydroxyethyl cellulose-glyoxal as the cell carrier.

### DETAILED DESCRIPTION OF THE PREFERRED EMBODIMENT

In accordance with the present invention, there is provided a new procedure of cell immobilization in a polymer matrix of acid-soluble chitosan brought to physiological pH with glycerol phosphate salt, then cross-linked with a bifunctional dialdehyde (glyoxal). The bifunctional dialdehyde is presented alone, or as a hemi-acetal intermediate conjugated with hydroxyethyl cellulose. This composition maintains high levels of cell viability, provided that the chitosan solution is sterile, and in liquid solution at isotonic and approximately neutral pH. For this purpose, acid-soluble chitosan may be sterilized by autoclave, or the crystalline powder salt form of chitosan sterilized by exposure to UV light prior to dissolving in water. The molecular mass of chitosan may be varied by autoclave-dependent hydrolysis resulting in a reproducible loss in viscosity, prior to adjusting to neutral pH with glycerol phosphate salt. In another embodiment, other phosphate buffers may be used that increase the chitosan solution to pH 6.5-6.8, without resulting in chitosan precipitation. The glycerol phosphate salt or phosphate buffer added brings the final osmolarity within physiologically-tolerated limits, or between 200 and 460 mOsm.

The pH dependence of chitosan cross-linking is strictly related to the percentage of free neutral amine groups available to participate in the cross-linking mechanism. Such a proportion of neutral amine-to-protonated amine groups is affected by the deacetylation level of the chitosan used. 95% deacetylated chitosan may be cross-linked at pH 5.0, whereas 80% deacetylated chitosan may only be cross-linked at a higher pH, above 6.0. The most favorable pH used to cross-link chitosan and simultaneously retain cell viability is generally above pH 6.5 at room temperature.

In accordance with the present invention there is provided a method for encapsulating and delivering live cells to a cell culture petri, ex vivo tissue, or in vivo within an implant, wound, organ space, or defect. Further, there is provided a method for co-gellation and sustained release of admixed proteins, such as IGF-1.

Cells are immobilized in neutral chitosan liquid solution with the aid of a cross-linking reagent. In the present embodiment, the cross-linking agent consists of glyoxal mixed with a polymer harboring reactive hydroxyl groups, such as hydroxyethyl ether. The combination of glyoxal-hydroxyethyl cellulose has much reduced toxicity to cells, because the presence of hydroxyethyl cellulose hinders the glyoxal aldehyde groups from reacting with the cell surface. The chitosan amine groups will preferentially attack the glyoxal reactive hydroxyl groups, resulting in a lattice of glyoxal-linked chitosan amine groups with hydroxyethyl cellulose interspersed throughout. The cross-linking agent may also consist of glyoxal mixed with physiological medium, which although less effective than glyoxal-hydroxy polymer cross-linkers in maintaining viability, can also sustain reasonable levels of viable cells in the final cross-linked chitosan gels.

The preferred physiological medium used to suspend the cross-linking agent is a nutrient medium suitable for cell culture, as opposed to simple buffered or unbuffered saline solutions.

The invention can be extended to encompass any cross-linking reaction whereby a hydroxyl-containing polymer is combined with a bifunctional reactive agent, and reacted with a poly-amine-containing polymer.

To immobilize cells homogenously, a cell pellet is completely resuspended in an aqueous solution of hydroxyethyl cellulose harboring glyoxal, or glyoxal in medium, then mixed with a neutral chitosan solution. The resulting mixture may be poured, or injected into the appropriate defect or mold, whereupon solidification occurs. The resulting gel has variable viscoelastisity, adhesivity, and stiffness, depending on the relative amounts of chitosan, glyoxal, and hydroxyethyl cellulose present in the mixture.

The injectable solution may also be used as a bulking agent or tissue sealant.

The present invention also includes, but is not limited to, the example of articular cartilage repair, where delivery of primary and/or passaged chondrocytes with said mixture to an articular cartilage defect will sustain cell viability, and permit proper cell differentiation and the synthesis and assembly of a dense mechanically functional articular cartilage extracellular matrix in situ. The invention includes intervertebral disc repair, where cross-linked gel, or cross-linked gel loaded with matrix-producing cells, is delivered to the damaged disc.

The injectable solution can also be previously mixed with growth factors, DNA, DNA-polymer complexes, liposomes, pharmacological agents, metabolic factors, antibodies, nutritive factors, angiogenic factors, or radioisotopes. To do so, these factors can be mixed with either the neutral chitosan solution, or with the cross-linking hydroxyethyl cellulose-aldehyde solution, prior to combining the chitosan and cross-linker.

In another embodiment, the cells may be suspended in a neutral chitosan solution, then mixed into hydroxyethyl cellulose neutral solution, with a range of chitosan/hydroxyethyl cellulose/cross-linker proportional volumes.

The hydroxyethyl cellulose needed to cross-link chitosan is preferably obtained by one of several methods from commercially available medium viscosity non-pharmaceutical grade hydroxyethyl cellulose. In routine industrial processing, hydroxyethyl cellulose is surface-treated with glyoxal to induce cross-links. The cross-linked hydroxyethyl cellulose is slow to dissolve in water, and therefore has reduced lumping. It is in these preparations that active chitosan cross-linker may be obtained. Pharmaceutical-grade hydroxyethyl cellulose, which has been treated to remove glyoxal, cannot be used to prepare active chitosan cross-linker.

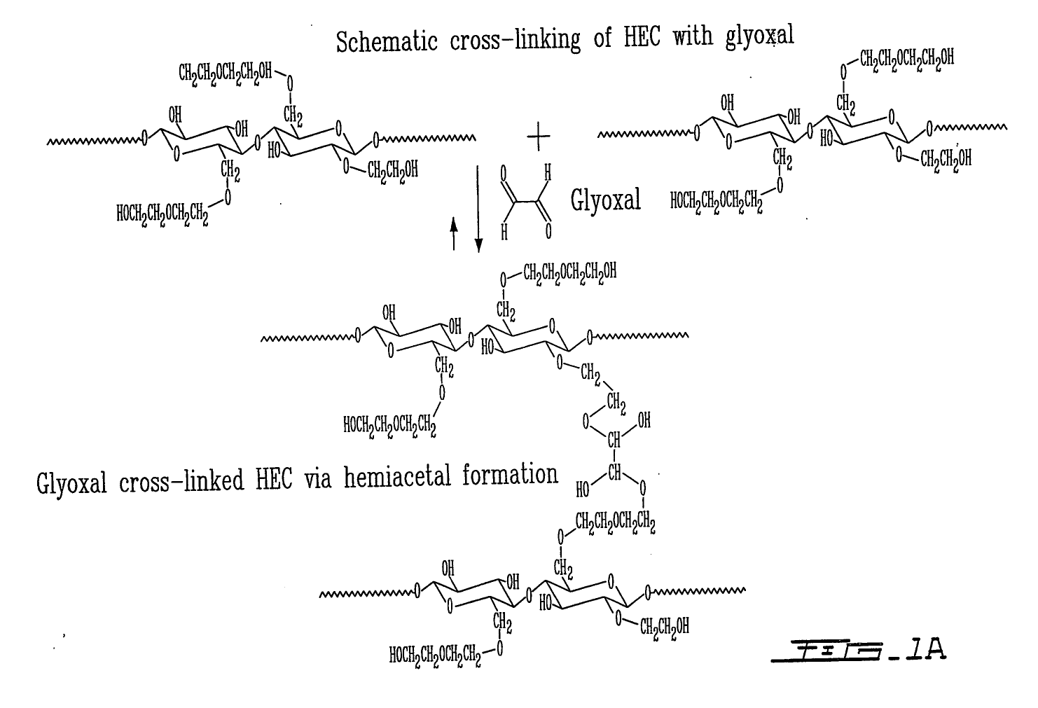

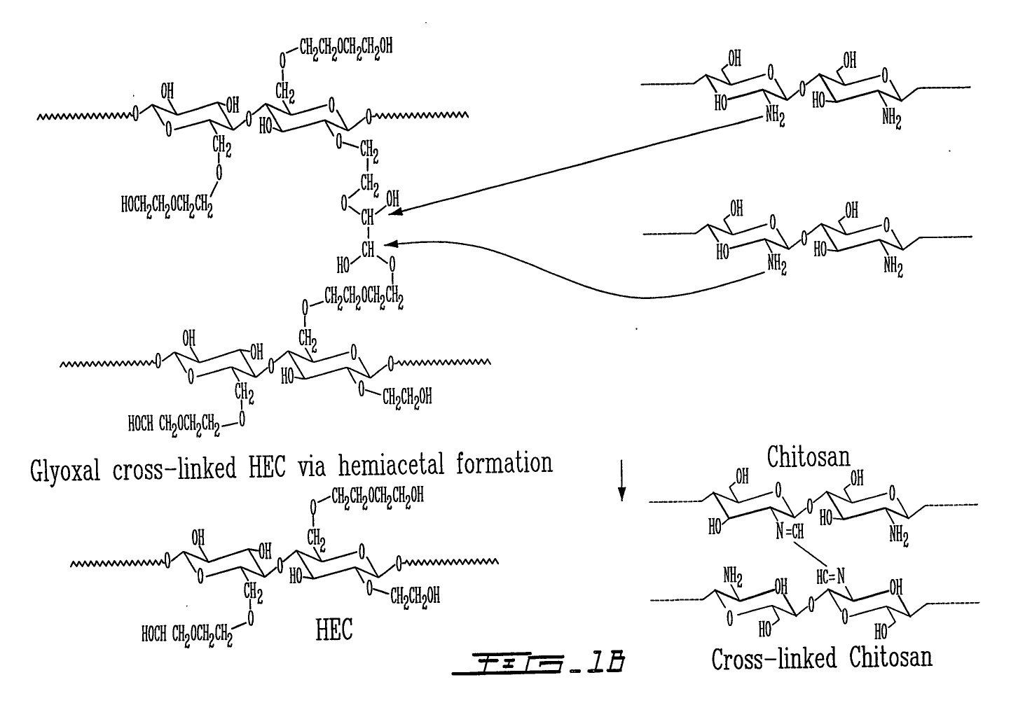

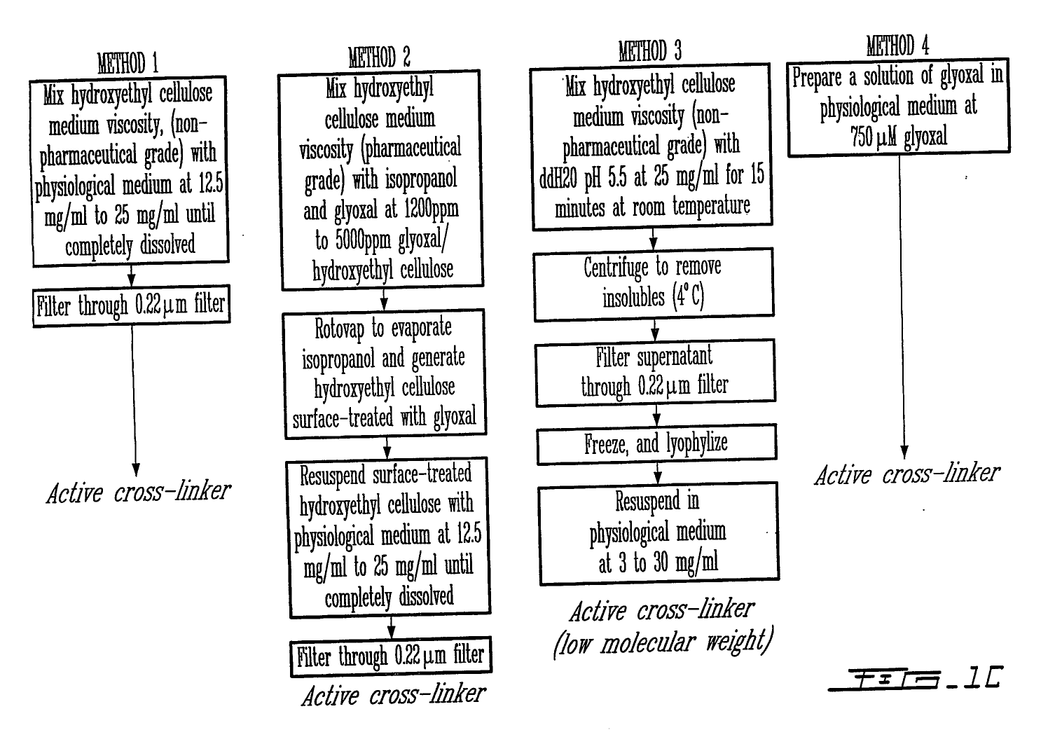

Several methods may be used to prepare cytocompatible cross-linker. By one method, certain types of medium viscosity hydroxyethyl cellulose (Fluka) can be dissolved completely to 25 mg/ml in aqueous solution at physiological pH. In one method (Method 4), a solution of 40% glyoxal (8.76M) is diluted to 750 μM in physiological medium. The resulting solution may be used as active cross-linker by mixing 1 part with 4 parts neutral chitosan., then sterilized by filtration through a 0.22 mm filter (method 1, FIG. 1C). FIG. 1C illustrates method 1, wherein hydroxyethyl cellulose of medium viscosity (3,400 cPa), non-pharmaceutical grade, from Fluka having slow dissolving time in water, has been cross-linked with glyoxal to retard the rate of hydration and to minimize lumping. If dissolved completely at 12.5 mg/ml to 25 mg/ml in physiological medium, the resulting solution may be sterile-filtered through a 0.22 μm filter, and used as active cross-linker by mixing 1 part filtered hydroxyethyl cellulose with 4 parts 1.5% neutral chitosan.

By another method, pharmaceutical grade hydroxyethyl cellulose is surface-treated with glyoxal and dried prior to dissolving in physiological media and filter sterilization (method 2, FIG. 1C). In Method 2 illustrated in FIG. 1C, hydroxyethyl cellulose of medium or low viscosity (pharmaceutical grade: below 500 ppm glyoxal or no glyoxal), is combined with 2500 ppm to 3500 ppm glyoxal in a polar solvent, and dried to generate hydroxyethyl cellulose surface treated with glyoxal. The resulting powder may be dissolved at 25 mg/ml in physiological medium, sterile-filtered, and used as an active cross-linker as described for Method 1 above. By another method, hydroxyethyl cellulose is mixed at 25 mg/ml with ddH₂0 for 15 minutes at room temperature, where the particles are resistant to water solubilization.

In method 3 of FIG. 1C, hydroxyethyl cellulose of medium viscosity, non-pharmaceutical grade, from Spectrum or Fluka, both have slow dissolving time in water. The water-soluble hydroxyethyl cellulose fraction is recovered, lyophilized, and the resulting solid resuspended in aqueous solution, which is physiological in pH and osmolarity (method 3, FIG. 1C). If the hydroxyethyl cellulose is mixed for 15 minutes in water, the aqueous phase which contains small molecular weight hydroxyethyl cellulose and in addition reactive glyoxal may be recovered by centrifuging out insolubles, and filtering through a 0.22 μm filter. The resulting solution may be concentrated and used to cross-link neutral chitosan by mixing 1 part (1 mg/ml to 30 mg/ml) water-soluble hydroxyethyl cellulose with 4 parts neutral chitosan.

Alternatively, glyoxal may also be diluted to that concentration present in surface-treated hydroxyethyl cellulose (near 0.001%) in physiological medium and rendered filter-sterile (method 4, FIG. 1D). In method 4 of FIG. 1C, a solution of 40% glyoxal (8.76M) is diluted to 750 μM in physiological medium. The resulting solution may be used as active cross-linker by mixing 1 part with 4 parts neutral chitosan. Some commercial hydroxyethyl cellulose powders will form a gel when dissolved completely at 25 mg/ml (Spectrum, Hercules). In this event, reactive cross-linker may only be obtained if the hydroxyethyl cellulose has been cross-linked with glyoxal, or another similar reagent, and if water-soluble material (containing low molecular weight cross-linked hydroxyethyl cellulose) can be extracted from slowly dissolving particles. Regardless of the method used to prepare the hydroxyethyl cellulose solution, once hydrated, the solution shall be protected from hydrolysis or conformational changes by frozen storage.

Active cross-linker can be purified from a low molecular weight fraction (below 1000 Da) of water-soluble hydroxyethyl cellulose from Spectrum. However, the more purified the cross-linker becomes, the more toxic an effect it has on cells. Therefore, the optimal cross-linking conditions for cell viability are those which use a cross-linking agent in the presence of an alternative polymer upon which the cross-linker may react, but which has less affinity for the cross-linker than does chitosan neutral amine groups. When the apparent toxic effect is due to co-purifying contaminants from the initial hydroxyethyl preparation, this toxicity may be partly avoided by using pure glyoxal at highly dilute concentrations in media.

The hydroxyethyl cellulose solution used to cross-link the chitosan-glycerol phosphate solution is preferably 0.5% to 98% the bulk mass of chitosan present in liquid solution. The solution is preferentially sterilized by filtration through a 0.22 mm filter. To those skilled in the art, it becomes obvious that any multifunctional reactive compound which may form reversible cross-links with a suitable polymer carrier could be used as a reduced toxicity, cytocompatible cross-linker for any amine-containing polymer, to entrap cells or bioactive molecules that are sensitive to incubation with the multifunctional compound alone.

Once prepared, the concentrated water-soluble hydroxyethyl cellulose is suspended in a physiological buffered solution, such as phosphate-buffered saline, Ringer's buffered lactate, cell culture medium such as Dulbecco's modified Eagle Medium, sterile 0.9% saline, or other preparations of cytocompatible nutrient medias used in cell culture. For delivery of some bioactive substances, chemicals, liposomes, radioisotopes, or pharmaceutical agents, the hydroxyethyl cellulose can be suspended in water or other conditions in order to combine completely with these materials prior to mixing with chitosan. For instances such as this, the chitosan does not necessarily need to be rendered to physiological pH, but instead, 95% deacetylated chitosan may be dissolved in a minimum amount of acid, and used at a pH of 4.0 to 5.5.

The present invention demonstrates that the gellation mechanism of neutral chitosan solutions using hydroxyethyl cellulose cross-linker may only occur when the hydroxyethyl cellulose solution has been previously combined with glyoxal in a surface treatment during routine large-scale industrial preparation. The present invention furthermore demonstrates that the cross-linking activity of hydroxyethyl cellulose is lost when glyoxal is eliminated by dialysis, or by other specific treatments used to remove glyoxal to generate a pharmaceutical grade product. It is shown in the present invention that at low concentrations (below 0.01%) glyoxal may be used to cross-link neutral chitosan solutions while maintaining cell viability, however initial cell metabolism (as an index of cell viability) of cells encapsulated in such glyoxal cross-linked gels is lower than that of cells encapsulated with hydroxyethyl cellulose-glyoxal. The kinetics of gellation shown in the examples of this invention are compatible with clinical use, from seconds to one hour, and permit the gellation and retention of gel with or without cells and/or medically active agents in a body cavity, petri dish, or open wound.

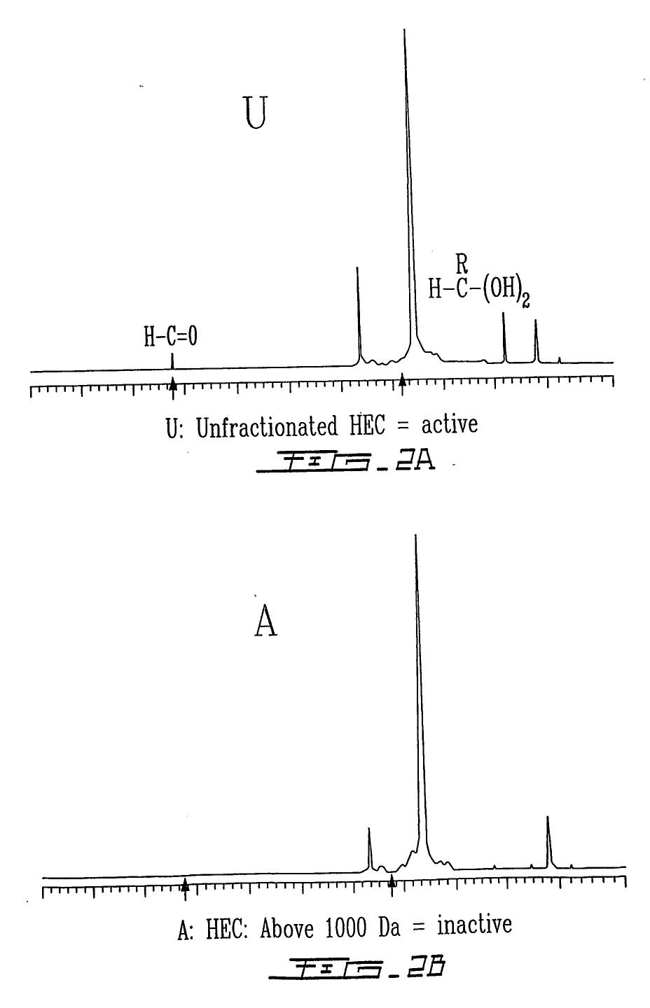

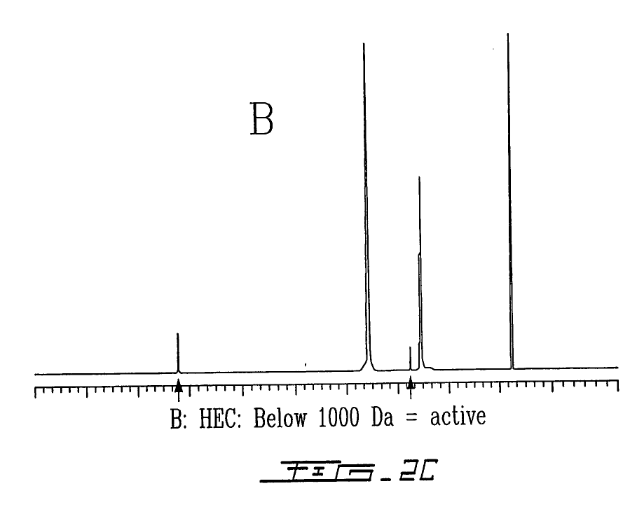

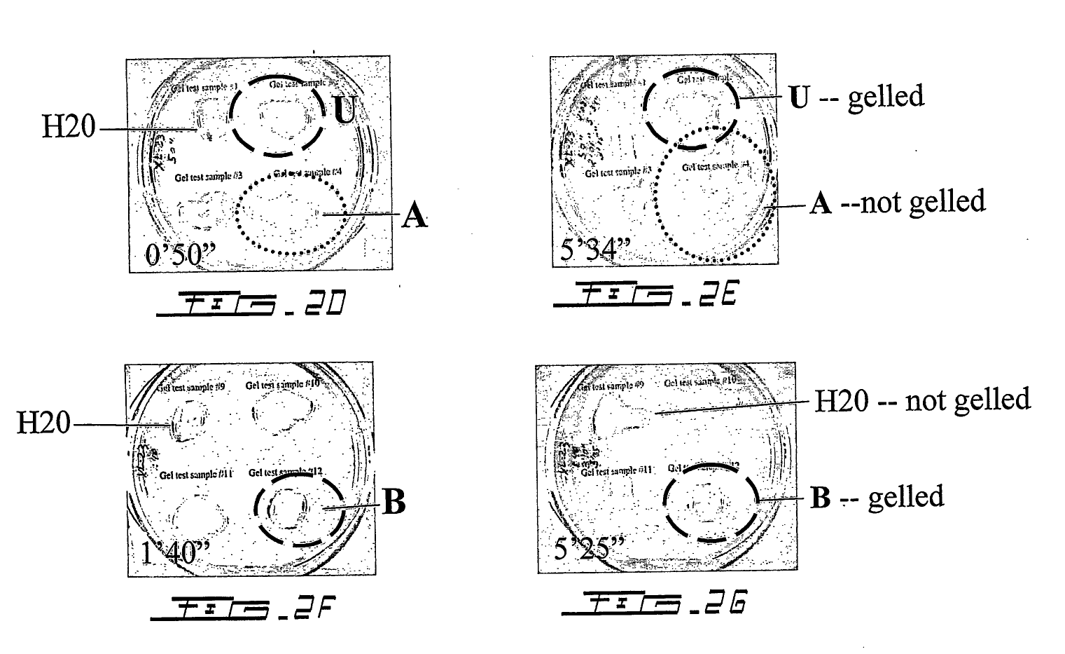

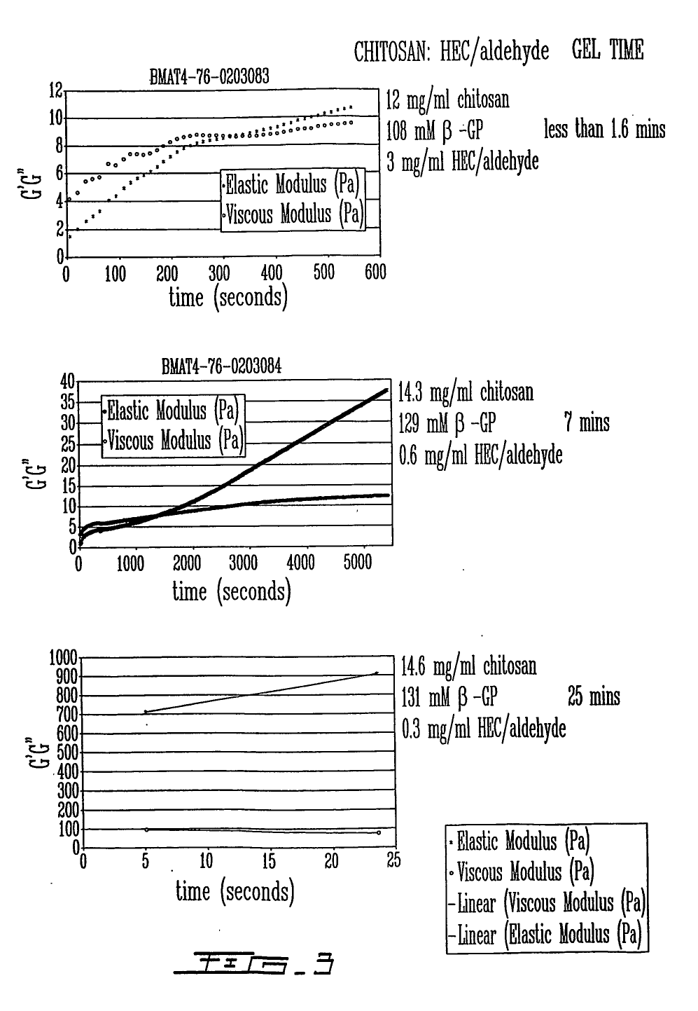

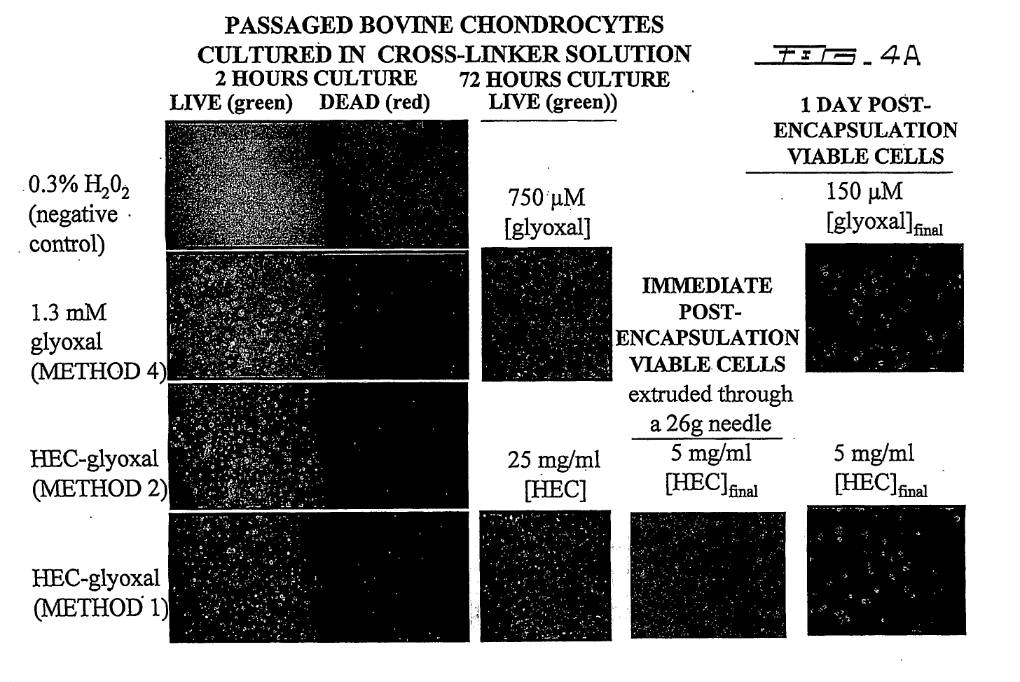

In FIGS. 2A to 2G, cross-linking activity correlates with those hydroxyethyl cellulose fractions containing aldehyde-like 1H-NMR peaks (peak at 8.3 ppm) and hemiacetal peaks (3.8 ppm). Cross-linker was prepared according to method 3 in FIG. 1C, and subsequently fractionated by ultrafiltration to collect fractions above and below 1000 Da. Each fraction was submitted to NMR analysis (upper panels). Each of the fractions was suspended at 7.5 mg/ml in ddH₂0, and mixed with neutral chitosan at 1 part hydroxyethyl cellulose fraction, 5 parts 1.5% neutral chitosan solution. The samples were deposited on a plastic petri, and tilted at timed intervals to demonstrate gellation (lower panels). Unfractionated, and the low molecular mass fraction (below 1000 Da) induced rapid gellation of chitosan within 5 minutes. Dialysed hydroxyethyl cellulose failed to gellify the chitosan, indicating that hydroxyethyl cellulose is not sufficient to cross-link chitosan under the test conditions. Both active cross-linking samples harbor peaks consistent with the presence of an aldehyde (8.3 ppm) and hemiacetal (3.8 ppm).

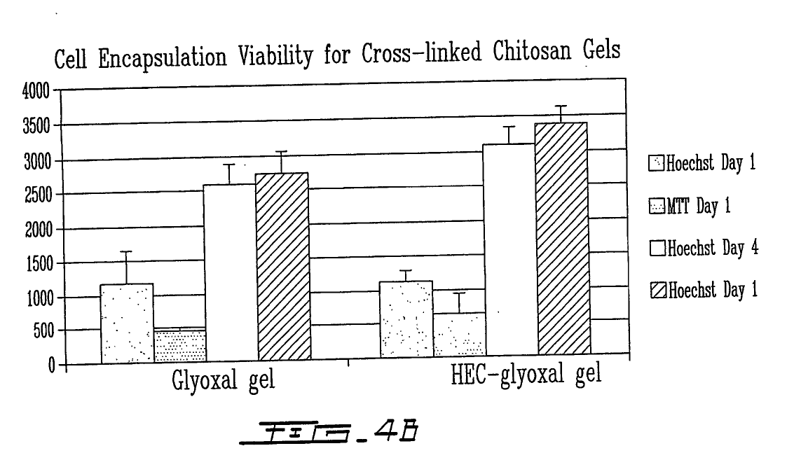

In FIG. 3, t=0 occurs 1.6 minutes after mixing. The results show a dose-dependency between gellation time, and hydroxyethyl cellulose-glyoxal concentration.

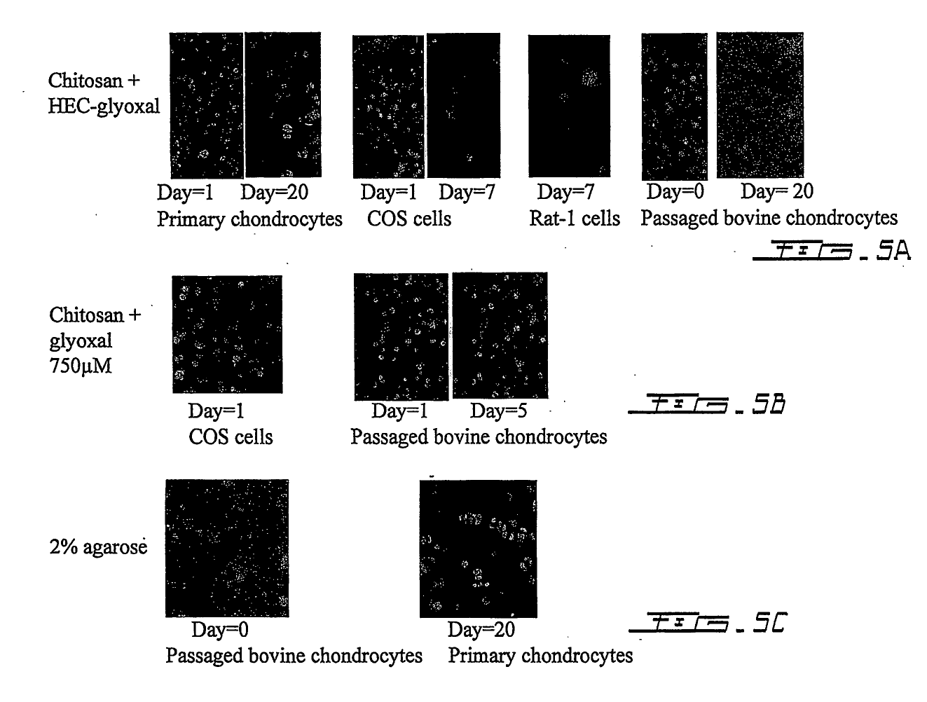

In FIG. 4A, high viability is maintained after encapsulation in cross-linked chitosan with glyoxal, or hydroxyethyl cellulose-glyoxal. FIG. 4A illustrates that the active hydroxyethyl cellulose-cross linker is cytocompatible. Cells incubated up to 72 hours in active cross-linker remain over 95% viable. Cells incubated in 0.3% peroxide for the same time period are 100% non-viable. After mixing with chitosan and injecting through a syringe with a 26-gauge needle, encapsulated cells in solid gel remain over 95% viable. After mixing with chitosan and pouring into a petri, encapsulated cells in solid gel remain over 95% viable after 1 day of culture.

In FIG. 4B, as shown by MTT assay on day 1 encapsulated cells, hydroxyethyl cellulose offers additional protection to cells immediately post-encapsulation. Cells encapsulated in chitosan gel using either glyoxal or hydroxyethyl cellulose-glyoxal are viable after encapsulation and proliferate in the gel. Cells show greater viability as measured by a metabolic MTT assay, at 1 day post-encapsulation when the active cross-linker is hydroxyethyl cellulose-glyoxal, compared to glyoxal cross-linker.

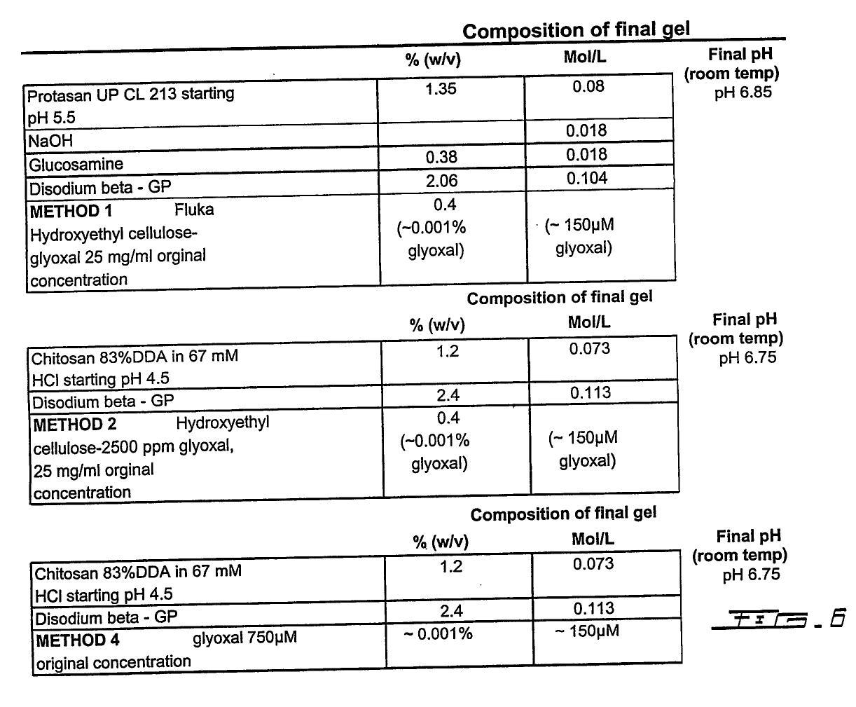

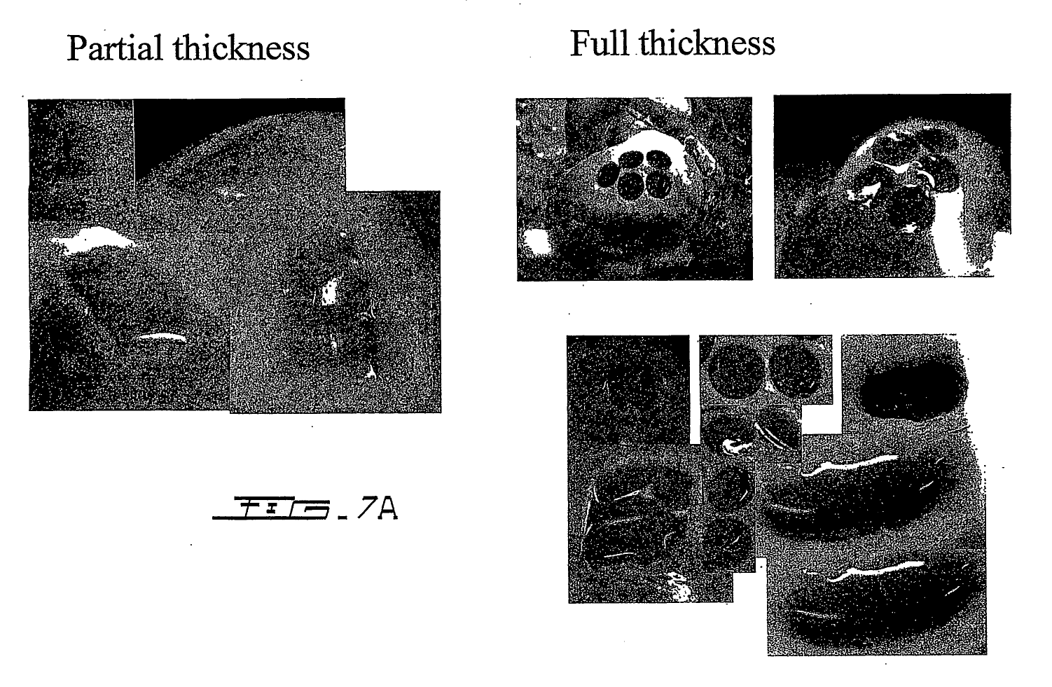

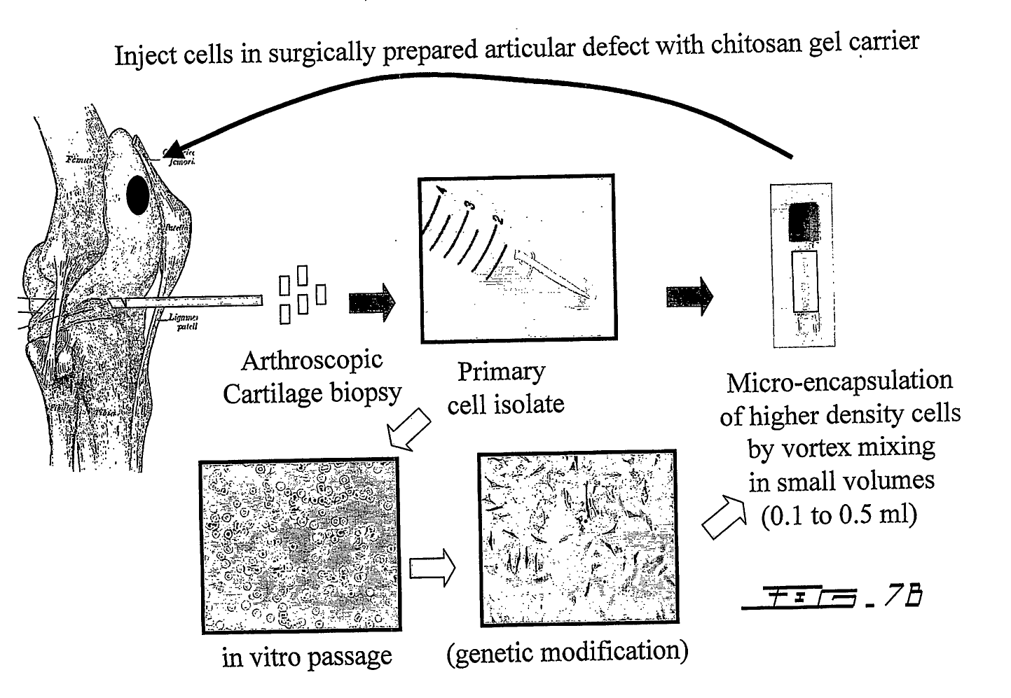

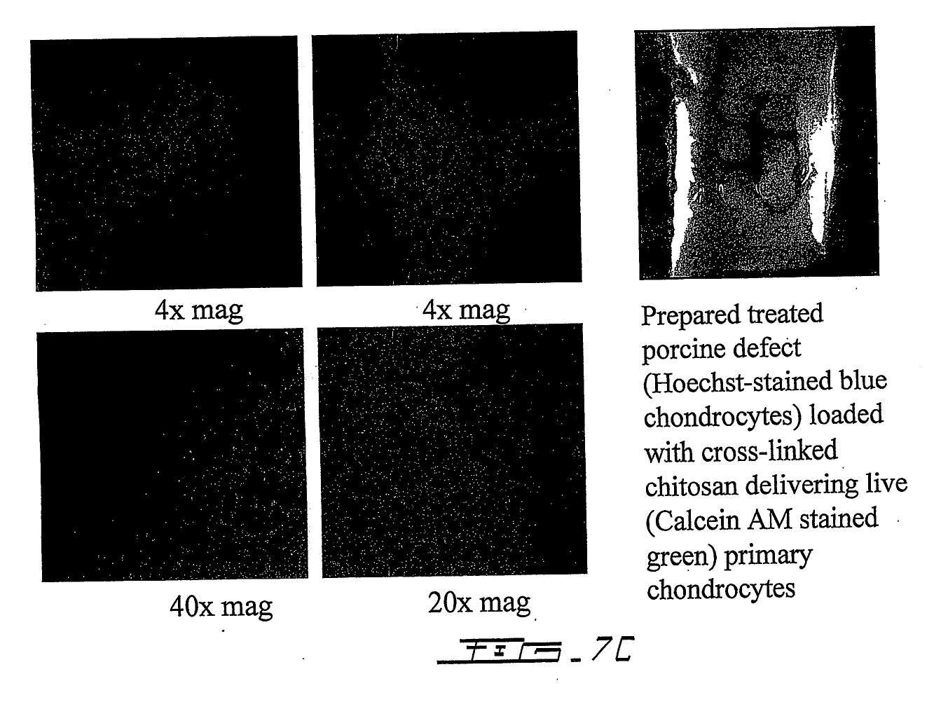

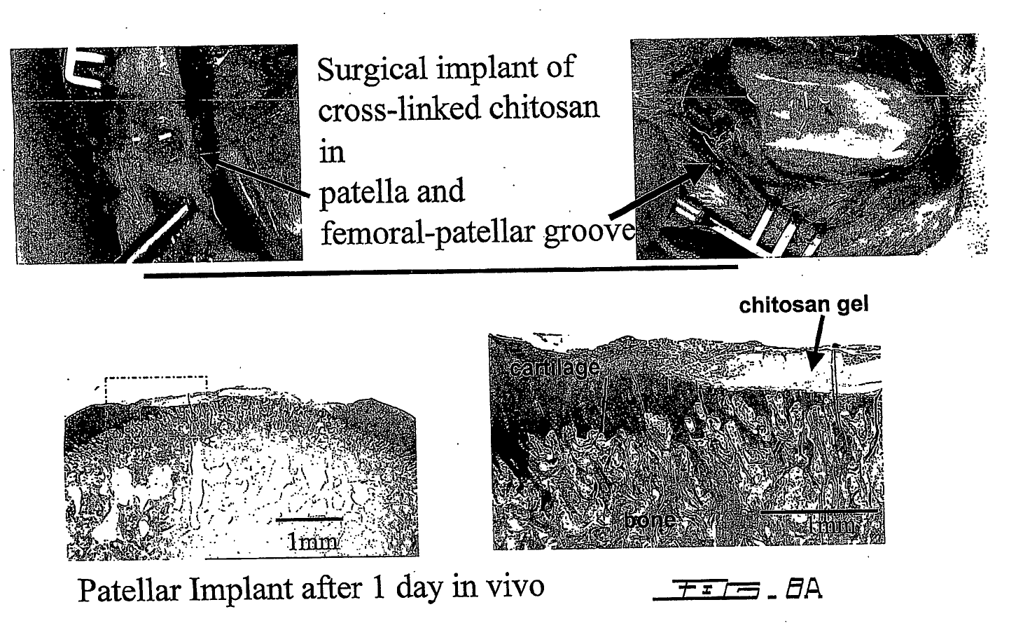

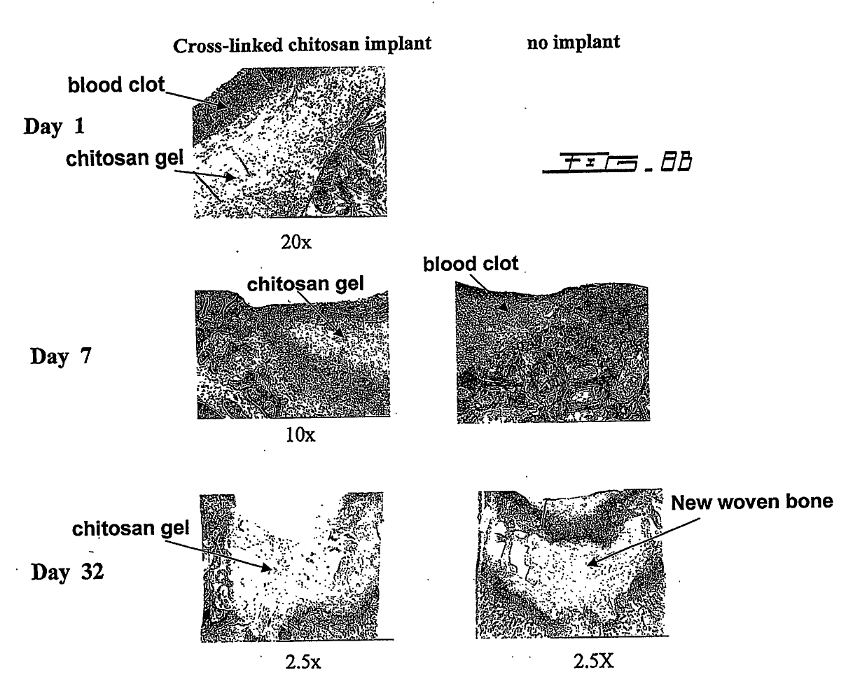

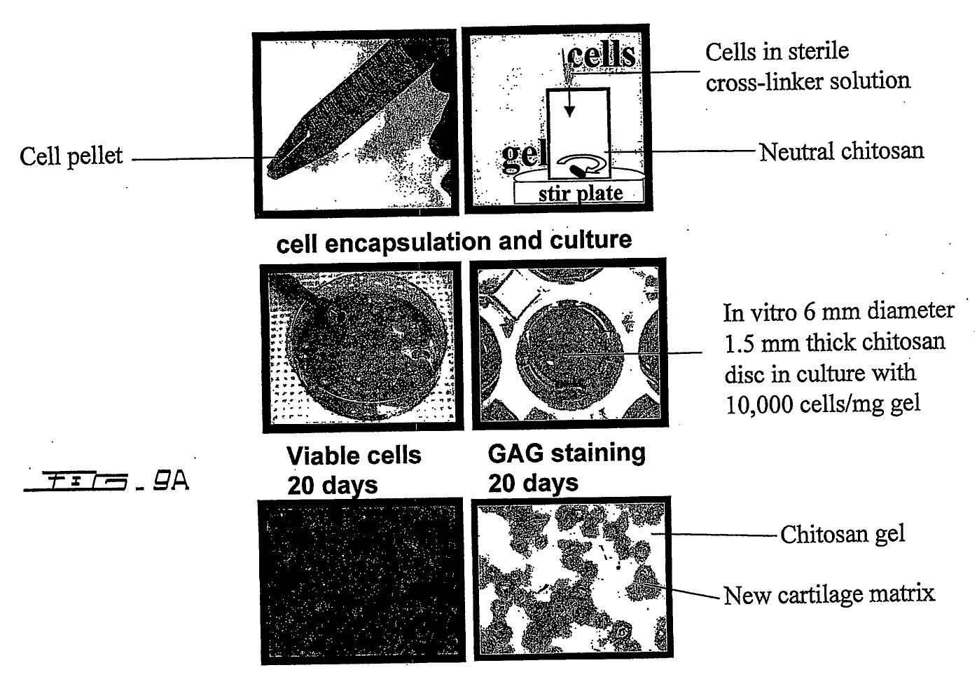

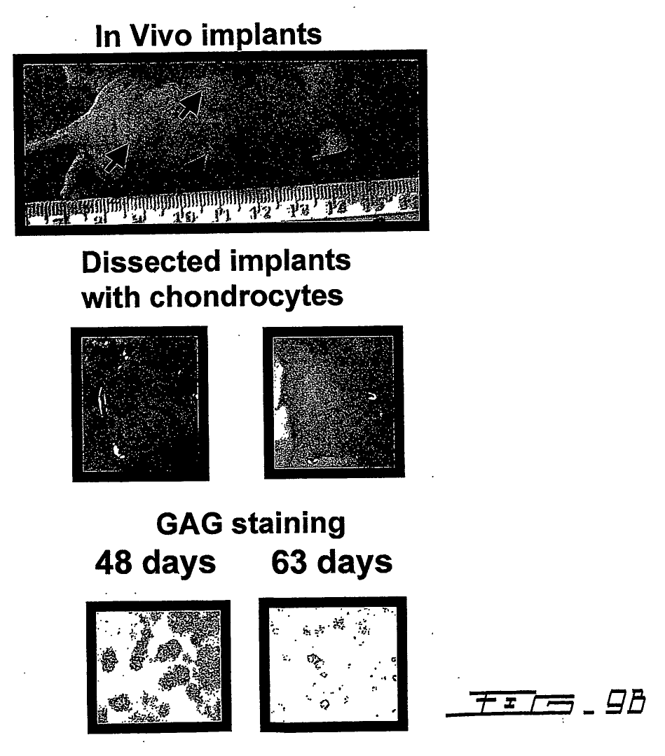

In FIGS. 5A to 5C, green is indicative of live cells and red is indicative of dead cells. As can be noted, FIG. 5A shows the persistence of a range of viable cell types cast in chitosan gels cross-linked with hydroxyethyl cellulose-glyoxal, including fibroblast cell lines Rat-1, COS, bovine primary chondrocytes, and bovine passaged chondrocytes at casting and after culture. FIG. 5B shows persistence of COS cell and passaged bovine chondrocyte cell viability in glyoxal cross-linked chitosan gels. FIG. 5C shows comparable viability of primary and passaged bovine chondrocytes cast in 2% low melting point agarose.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition for immobilizing and encapsulating viable and functional cells or bioactive substances comprising: a) a liquid polysaccharide solution of isotonic neutral chitosan; and b) a cross-linking solution consisting of a bifunctional or multifunctional, aldehyde or aldehyde-treated hydroxyl-containing polymer dissolved in physiological media.

2. The composition of claim 1, wherein the cross-linking solution consists of a bifunctional or multifunctional cross-linker and a hydroxylated polymer of appropriate ratio and molecular mass such as to permit the hydroxylated polymer to remain liquid in solution.

3. The composition of claim 1, where the cross-linking solution consists of glyoxal, or glyoxal-treated hydroxyethyl cellulose dissolved in a physiological medium.

4. The composition of claim 1, wherein the composition comprises: a) 0.5 to 5.0% by weight chitosan, or chitosan derivative, or poly-amine containing polymer; and b) 0.01 to 5.0% by weight hydroxyethyl cellulose, wherein said solution form a gel between temperatures of 4 C and 42 C, said gel providing a physiological environment for maintaining viability of cells.

5. The composition of claim 4, further comprising:
c) 0.0001-3% glyoxal.

6. The composition of claim 4, wherein the composition forms a gel within seconds to several hours after mixing (a) and (b).

7. The composition of claim 5, wherein the composition forms a gel within seconds to several hours after mixing (a), (b) and (c).

8. The composition of claim 4, wherein the solution forms a gel between temperatures of 20 C and 42 C.

9. The composition of claim 1, wherein the chitosan is dissolved in dilute acid and mixed with 1.0 to 2.5% by weight of a salt of polyol consisting of mono-phosphate dibasic salt, or mono-sulfate salt.

10. The composition of claim 9, wherein said mono-phosphate dibasic salt is mono-phosphate dibasic salt of glycerol.

11. The composition of claim 9, wherein mono-phosphate dibasic salt of glycerol is selected from the group consisting of glycerol-2-phosphate dibasic salt, sn-glycerol 3-phosphate dibasic salt and L-glycerol-3-phosphate dibasic salt.

12. The composition of claim 1, wherein chitosan is further mixed with phosphate buffer and salt.

13. The composition of claim 1, further comprising a biologically active factor.

14. The composition of claim 13, wherein the biologically active factor is selected from the group consisting of cells, a hormones, a drug, DNA, a bulking agent, a growth factors, a DNA, DNA-polymer complex, liposomes, a pharmacological agent, a metabolic factor, an antibody, a nutritive factor, an angiogenic factor, and a radioisotope.

15. The composition of claim 14, wherein said cells are live cells.

16. The composition of claim 14, wherein the cells are nucleus pulpopus, annulus fibrosis, or a mixture thereof.

17. The composition of claim 14, wherein the cells are embryonic stem cells or stem cells derived from a tissue selected from the group consisting of bone marrow, adipose, muscle, brain, skin, liver, vascular smooth muscle, endothelium, blood, or placenta.

18. The composition of claim 14, wherein the cells are primary cells, differentiated cells, genetically modified cells, hybridomas, immortalized cells, transformed cells, tissue fragment cells, organelles, or a mixture thereof, nucleated cells, enucleated cells, germ cells, platelet cells, matrix vesicles, cell vesicles, demineralized bone paste, bone chips, cartilage fragments, or cell fragments or tissue fragments.

19. The composition of claim 14, wherein the cells are autologous cells, allogeneic cells or xenogeneic cells.

20. The composition of claim 14, wherein the biologically active factor is a cell attachment factor selected from the group consisting of fibrinogen, fibrin, fibronectin, hyaluronic acid, heparin, collagen, polylysine, polyornithine, receptor-binding cyclic peptide, receptor-binding protein.

21. The composition of claim 14, wherein the biologically active factor is an enzyme, a growth-factor or a growth factor-immobilized substance.

22. The composition of claim 14, wherein the biologically active factor is a plasmid DNA in the form of liposomes, a lipid complex, a chitosan complex, a poly-lysine complex, a DEAE dextran complex.

23. The composition of claim 14, wherein the biologically active factor is a vaccine.

24. The composition of claim 23, wherein the vaccine comprises an infective viral particle.

25. The composition of claim 14, wherein the biologically active factor is a nutritive or metabolic factor.

26. The composition of claim 25, wherein the a nutritive or metabolic factor is a lipid, amino acids, and a co-factor selected from the group consisting of cholesterol, glutamin, glucosamine, ascorbic acid, pyruvate, and lactate.

27. The composition of claim 14, wherein the biologically active factor is at least one element selected from the group consisting of peripheral blood, bone blood, cord blood, a blood product, blood-borne cells, serum, platelets, platelet-rich plasma, fibrinogen, a clotting factor, and a blood-borne enzyme.

28. The composition of claim 14, wherein the biologically active factor is an osteogenic substance.

29. The composition of claim 28, wherein the osteogenic substance is a member of the bone morphogenetic protein family selected from the group consisting of TGF-p1, BMP-2, BMP-6, BMP-7, or a mixture thereof.

30. The composition in claim 1, wherein hydroxyl-containing polymer is polyvinyl alcohol, dextran, linked with a bifunctional reactive aldehyde.

31.-40. (canceled)

41. A method for repairing soft tissue, said method comprising the step of administering the composition of claim 1 at the site of a soft tissue in need of repair of a patient.

42. A method for repairing or resurfacing a damaged cartilage, said method comprising the step of administering the composition of claim 1 in or around a cartilage in need of repair or resurfacing of a patient.

43. A method for repairing a meniscus of a patient, said method comprising the step of administering the composition of claim 1 at the site of the meniscus in need of repair.

44. The composition of claim 3, where the physiological medium comprises cell nutrients selected from the group consisting of glucose, amino acids, and vitamins, or a combination thereof, at isotonic and neutral pH.
